(19) United States
(12) Patent Application Publication
(10) Pub. No.: US 2019/0008896 A1
(43) Pub. Date: Jan. 10, 2019

(54) Methods and Systems for the Prevention, Treatment and Management of Disease and Effects of Aging Via Cell-to-Cell Restoration Therapy Using Subcellular Transactions

(71) Applicant: Richard Postrel, Miami Beach, FL (US)

(72) Inventor: Richard Postrel, Miami Beach, FL (US)

(21) Appl. No.: 15/952,233

(22) Filed: Apr. 12, 2018

Related U.S. Application Data
(60) Provisional application No. 62/529,551, filed on Jul. 7, 2017.

(51) Int. Cl. A61K 33/40; A61K 38/18; A61K 45/06; A61P 3/00

(52) U.S. Cl. CPC A61K 33/40; A61K 38/1808; A61K 38/1825; A61K 31/352; A61K 45/06; A61P 3/00; A61K 2300/00; A61K 38/1841

(57) ABSTRACT

This invention describes an cellular and subcellular “social network” at a biomolecular level that provides systems and methods for life extension, disease prevention and cellular rejuvenation through the transfer or exchange of biochemical, electrophysiological and sub-cellular biomolecular constructs and/or their assemblies, either in their totality or in part as carriers of material and/or information through interactions and continuous intercourses between neighboring cells. First responder cells are engineered or primed to support such conveyances including, but not limited to: organelles, macromolecules, lipids, ions, etc. from cells able to dispose of said biomaterials, etc. to cells requiring or standing to benefit therefrom. Among other advantages, these systems and methods provide increased capacity for managing intact mitochondrial (switching) mechanisms for the restoration or improved functionality of compromised cells. As cells or cellular clusters signal or are recognized by neighboring cells, cellular assemblage(s) or biomass, they receive mitochondria that are tagged, selected or engineered for cyto-enhancement. First responder cells may be cells native to the organism in situ; cells native to the organism, but removed and cultured or modified in growth media, optionally synthetically induced to express improved functionality, and returned per ipsis or combined with restorative media to aid healing. Additional immunologically compatible cells cultured from sources other than those native to the organism may be provided as an aid to healing. This process may be activated by using chemical, physical or harmonic activators or a combination thereof.

Life is a complicated interaction of physics (movement—including temperature and sound, light, mass attraction, electricity, magnetism), chemistry (interaction of electrons amongst atoms putting together molecules, etc. and these larger structures, including their functionalities), and biology (melding of chemistry and physics to produce organisms capable of making like organisms [self-replication]). Biology is the science of life, a process embodying sentience, sharing of experiences, remote sensing and transfer of experiences and, as evidenced in humans, ability to understand and improve life itself.

Even in single celled organisms, the molecules in the cells apply physics (movement, temperature, diffusion) and chemistry (making and breaking chemical compounds) to deviate from chemical equilibrium to maintain a living status of dynamic equilibrium. Cells comprise specialized chemical molecules that act as catalysts on other chemicals within the cell to move atoms and chemical energy from one molecule to another molecule in a manner that at that time is useful for continuing the cell's metabolism in preparation for a series of subsequent reactions.

In the more complex (eukaryotic) single cell organisms and larger multi-cell organisms, each cell is subdivided into specialized compartments including, but not limited to: the nucleus for storing and transcribing nucleic acids, the endoplasmic reticulum (ER) for translating nucleic acids into proteins, golgi for processing and packaging the proteins from the ER, mitochondria for providing the chemical energy enabling normal operations of the rest of the cell, etc. The mitochondrion, along with the chloroplast of photosynthesizing plants, is remarkable in that these organelles maintain their own separate store of nucleic acid as a genome that encodes organelle dedicated genetic material that is transcribed and translated within the membranes of the organelle.

Nucleic acid in the nucleus and in genomes of these special organelles is an ordered polymer of four similar nucleotide molecules, the precise order of which provides instructions for making the proteins that help provide structure and are important catalysts for maintaining life. When, for example, nucleic acid, in the genome of the mitochondrion is disrupted or mutated, RNA is not properly transcribed (copied from the DNA). Since RNA molecules are required to synthesize the proteins encoded in the genome, the result is severe degradation of mitochondrial function. Mitochondria comprise a double membrane that surrounds inner matrix cristae. The membranes provide a barrier to and control the flux of most internal cell components. Damage to or malformation of the mitochondrial membranes, a build-up of denatured proteins in solution or in the membranes, and/or toxins in one or more mitochondrial compartments will comprise the function of the affected mitochondrion and the efficiency of the surrounding cell.

Mitochondria are often called the “powerhouse” or battery of the cell. A eukaryotic cell typically has multiple interconnecting mitochondria, the number and density of which increasing in cells with higher rates of metabolism (chemical reactions). The molecule adenosine triphosphate (ATP) functions as a predominant energy store and energy donor in the cell. The majority of the ATP used by the eukaryotic cell results from series biochemical processes that are carried out by and in their mitochondria. Within the cell, mitochondria tend to locate themselves in regions with higher activities. Each cell has mechanisms to control mitochondrial dividing to make new mitochondria when needed and degradation to recycle mitochondrial material when there is a greater need elsewhere. By balancing these processes the cell controls the number of mitochondria, their size and shape, and the metabolic level of the cell. Cells may contain hundreds to hundreds of thousands mitochondria. Cells also control movement of mitochondria within the cell so that substrates and products can be efficiently delivered. Assisting the cells and the organism containing the cell to optimize these mitochondrial activities will be found useful in optimizing the cell's and the organism's metabolism and health.

Modern mitochondria have many similarities to some modern prokaryotes. Many believe that mitochondria represent a symbiosis arising from an early bacterium infecting and then becoming symbiotic with the host cell. Mitochondria are split between the two daughter cells when eukaryotic cells divide. Even though they have diverged significantly since the ancient symbiotic event mitochondrial structure is much like that of bacteria. For example, the inner mitochondrial membrane contains electron transport proteins like the plasma membrane of prokaryotes, and mitochondria also have their own prokaryote-like circular genome. One difference is that these organelles apparently have lost most of the genes once carried by their prokaryotic ancestor.

Although present-day mitochondria do synthesize a few of their own proteins (14 in humans), the vast majority of the proteins mitochondria require to carry out their reactions origin proteins act in concert to carry out functions specific to the mitochondrial membranes, matrix and intermembrane space. These reactions include pathways essential for building extramitochondrial cell components, for example, amino acids for proteins and nucleotides for the DNA nucleic acid of our genomes.

Thus, since the majority of the mitochondrial proteins, but not all mitochondrial proteins are encoded and translated in cell nucleus, the mitochondrial based abnormalities or dysfunctions may be rooted in the mitochondrion or they may have cellular extra-mitochondrial origination. And a disturbance or dysfunction of any of these interconnecting pathways can compromise mitochondrial function which detrimentally impacts cellular energy metabolism and accordingly the health of the entire organism.

We humans are complex organisms comprising thousands of cell types. A major driver of growth process involves stem cells (cells that are capable of dividing into several types of cells). When conditions are right these cells divide to produce a daughter cell differentiated to carry out the task of that portion of tissue and another stem cell. The daughter cells specialize in one or more of the tasks undertaken by that tissue. These differentiated cells have emphasized the transcription and translation of the genes associated with that differentiated cell type and have lost the ability to express other genes, especially genes for mitosis. These differentiated cells will not divide to pass on genetic status, including any mutations they might develop, to a next cell in the lineage. These cells will merely continue to function for better or worse so long as the nutrition provided to them and signals and support from other cells permits.

Metabolism in these differentiated daughter cells and the generally quiescent stem cells continues to produce ATP—the chemical energy packet used to make the macromolecules needed by the cell, and reactive oxygen species (ROS) as a byproduct of metabolism. ROS occur from incomplete oxidation in the electron transport chain (ETC) of the ATP generating complexes in mitochondrial membranes, are necessary in small quantities for generating chemical signals and for several specific oxidation reactions. So long as the cells continue to metabolize, they will leak electrons and ROS from the intracellular damage will proceed at least at a basal level. Accumulated metabolic damage from all internal and external sources eventually will compromise the cell to the extent that it is a drain on rather than a benefit to the organism. Under these circumstances the inefficient cells often self-cull using a process called apoptosis. A combination of the signals from outside the cell reaching the plasma membrane and the signals from within the cell interact to control a cell's apoptotic prospects and to control that cell's death. Apoptosis is to be distinguished from cell death resulting from injury. Injured, non-apoptotic cells can rupture on impact or when they cannot maintain the electrochemical gradient across the plasma membrane (an ATP dependent process) they absorb water, swell and burst. Physical or electrochemical rupture leaks the cell's contents and attracts lymphocytes as part of a damaging inflammatory response.

These individually apoptosing cells will by their nature as individuals not be present in large clonal numbers. The different cells will have different levels of damage and will undergo apoptosis at a time when conditions in and around that individual cell signal the cell to initiate apoptosis. In these circumstances, an individual cell's status may be appreciated by neighbor cells, but the status of the specific molecular deficiencies of any individual cell will be averaged merely as background noise. The consistent turnover of damaged cells is one process used to maintain a healthy metabolism.

Humans, like eukaryotic organisms generally, rely on their mitochondria to produce adenosine triphosphate (ATP) as a common store and provider of chemical energy to be used to empower other chemical reactions. In a mitochondrion, at the mitochondrial inner membrane, electrons from NADH and succinate are transported molecule to molecule by the Electron Transport Chain (ETC) to eventually combine with oxygen, which, when it accepts the electrons, is reduced and reacted with hydrogen atoms to make water. Along the way, the ETC comprises enzymatically transferring electrons through several electron donor and receptor compounds in series, eventually depositing the transported electrons with oxygen. Passing electrons from donor to acceptor releases energy in the form of a proton (H⁺) across the mitochondrial membrane. This electrochemical gradient has the ability to do work. This ETC mediated process is known as oxidative phosphorylation and results in production of adenosine triphosphate, aka, ATP.

In addition to the classical duty of the mitochondrion to supply energy, the mitochondrion also is intrinsically involved in processes of cell signaling, cell growth and proliferation, cell metabolism and cell death. Given the diverse activities of mitochondria it is not surprising that genetic and/or metabolic alterations in mitochondria appear to be involved in a plethora of diseases, including several cancers. Mitochondria are also intracellular signaling organelles. They mediate bidirectional intracellular information transfer: anterograde (from nucleus to mitochondria, endoplasmic reticulum to mitochondria, cytoplasmic membrane to mitochondria, peroxisome to mitochondria, etc.) and retrograde, the reverse. Mitochondria also act as messengers between cells, for example participating in a facet of an offshoot of our juxtacrine signaling system with cytonemes, filopodia, etc. The mitochondrial genome (mtDNA) and even whole mitochondria are mobile and can transfer their composition of matter, their functionalities and their stored information to an adjacent cell, generally one plagued with malfunctioning or nonfunctioning mitochondria.

Mitochondria are excellent movers of electrons as they oxidize many different molecular species in the chemical reactions that are catalyzed by one or more of the various mitochondrial metabolic complexes. In these metabolic processes, mitochondria also produce ROS, powerful oxidizing compounds that may be beneficial when oxidizing an appropriate substrate, but are quite damaging when they react on unintended molecules, e.g., a segment of mitochondrial genome—damaging the DNA; or on peroxidizable lipids initiating damaging peroxidation cascades. When ROS cause free radical formation that is not properly attenuated or scavenged, the resulting free radical cascade a series of spontaneous reactions where an unpaired electron is passed from molecule to molecule can be extremely damaging to a multiplicity of molecular components in the mitochondrion and throughout the cell.

Normally, about 1 or 2% of the O₂ consumed is incompletely reduced and becomes an ROS. This results mostly from a small fraction of reducing equivalents diverting off from complex I or complex III of the mitochondrial ETC to form the free radical superoxide anion, O₂⁻. The electron that provides this diatomic oxygen molecule with its negative charge causes an unstable configuration resulting from the odd number of electrons—and one electron not having another electron to pair with. Molecules with unpaired electron are extremely reactive and can pass the unpaired electron to another molecule in a destabilizing cascade. Mitochondrial superoxide dismutase then converts the O₂⁻ to H₂O₂ (not a free radical), but which can be reduced further to another highly reactive radical OH (hydroxyl radical), a hydroxide ion stripped of an electron.

ROS molecules themselves are important signaling molecules that mediate changes including, but not limited to: cell proliferation, differentiation, transcription, etc. Improper or exuberant ROS activity, sometimes termed oxidative stress, has the ability to initiate lipid peroxidation cascades that will corrupt lipid biomembranes and damage membrane and other intracellular proteins, lipids, and nucleic acids, including DNA. The DNA of the mitochondrial genome is especially susceptible to ROS damage. It is quite close to where ROS is produced (i.e., the mitochondrial inner membrane and the ETC), and it has no introns or protective histones and only a limited capacity for DNA repair. Oxidative stress therefore impedes mitochondrial function directly in the membranes, in the enzyme complexes, in membrane transport; and indirectly in disruption of mtDNA. Severe or prolonged oxidative stress will lead to irreversible oxidative damage and consequent cell death. Oxidative stress in mitochondria over a lifetime of metabolic reactions compromises the mechanisms of that metabolism with evidence apparent in several aging processes.

The health and function of mitochondria can be improved by, for example, nutrition targeted for those compromised mitochondria's current status and the current status of the surrounding cells, for example by providing to the cell and its mitochondria one or more, substrates, cofactors, and/or enzymatic activity modulators including, but not limited to: Riboflavin (B₂), L-creatine, CoCQ₁₀, L-arginine , L-carnitine, vitamin C, cyclosporin A, manganese, magnesium, carnosine, vitamin E, resveratrol, α-lipoic acid, folinic acid, fulvic acid, dichloro-acetate, succinate, prostaglandins (PG), prostacyclins, thromboxanes, prostanoic acid, 2-arachidonoylglycerol, NSAIDS, melatonin, cocaine, amphetamine, AZT, mitophagic controlling compounds, glutathione, β-carotene and/or other carotenoids, etc. But when mitochondria are severely stressed more robust actions may be necessary for cell survival.

However, some supplements or intended therapeutic compounds will have the opposite effect. Some common mitochondrial perturbations that have been recognized to result from ingesting supplements or taking drugs include, but are not limited to—they will: deplete numbers of mitochondria; increase ROS secretions from mitochondria; slow oxidative phosphorylation by mitochondria; decrease ATP production by mitochondria; compromise the integrity of the mitochondrial membrane causing excess heat. Antibiotics are especially at risk to mitochondrial health. This should not be unexpected given the similarities shared between mitochondria and bacteria. Antibiotics including, but not limited to: fluoroquinolones, macrolides, clindamycin, chloramphenicol, sodium azide, rifampin, tetracycline, azithromycin, roxithromycin and linezolid inhibit mitochondrial functions.

Bactericidal antibiotics, e.g., quinolones, aminoglycosides and β-lactams (including, but not limited to: penicillin derivatives (penams), cephalosporins (cephems), monobactams, and carbapenems) affect mitochondrial metabolism though interference with the oxidative phosphorylation/ATP production at the mitochondrial inner membrane with resultant increase in ROS release, decreased ATP availability and decreased O₂ consumption. Both mitochondrial and nuclear DNA show antibiotic exposure related damage—with the minimal mitochondrial DNA repair mechanisms compounding coding errors in the organelles.

Quinolones include, but are not limited to: flumequine, nalidixic acid, oxolinic acid, piromidic acid, pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, besifloxacin, clinafloxacin, gemifloxacin, sitafloxacin, trovafloxacin, prulifloxacin, etc.

The penicillin family of antibiotics includes, but is not limited to: amoxicillin, ampicillin, bacampicillin, carbenicillin, cloxacillin, dicloxacillin, flucloxacillin, mezlocillin, nafcillin, oxacillin, penicillin g, penicillin V, phenoxymethylpenicillin, piperacillin, pivampicillin, pivmecillinam, ticarcillin, etc.

Cephalosporins include, but are not limited to: cephacetrile, cefadroxyl, cefalexin, cephaloglycin, cephalonium, cephaloradine, cephalothin, cephapirin, cefatrizine, cefazaflur, cefazedone, cephazolin, cephradine, cefroxadine, ceftezole, cefaclor, cefonicid, cefproxil, cefuroxime, cefuzonam, cefmetazole, cefotetan, cefoxitin, carbacephems, loracarbef, cephamycins:, cefbuperazone, cefmetazole , cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, oxacephems, moxalactam, cefclidine, cefepime, cefluprenam, cefoselis, cefozopran, cefpirome, cefquinome, oxacephems, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, nitrocefin, etc.

Monobactams including, but not limited to: aztreonam, etc.

Carbapenems include, but are not limited to: imipenem, imipenem/cilastatin, Doripenem, Meropenem, Ertapenem, etc.

Aminoglycosides including, but not limited to: amikacin, gentamicin, kanamycin, neomycin, netilmicin, paromomycin, streptomycin, tobramycin, etc., inhibit mitochondrial protein synthesis by two modes of action. They bind a ribosomal subunit to interfere with peptide elongation. And when tRNA binds the ribosome, an aminoglycoside induced mRNA-tRNA reading mismatch is a frequent occurrence resulting in mis-sequenced, dysfunctional proteins and/or prematurely truncated proteins with no activity or improper activity.

The tetracycline family of antibiotics including, but not limited to: tetracycline chlortetracycline, oxytetracycline, demeclocycline, lymecycline, doxycycline, meclocycline, methacycline, minocycline, sarecycline, tigecycline, rolitetracycline, omadacycline, etc.; as well as the antibiotic chloramphenicol representing another class of antibiotic, preferentially target mitochondrial translation by binding one of the subunits of the mitochondrial ribosome. This extra molecule bound on the ribosome inhibits or prevents amino-acyl tRNA binding to the ribosome or formation of the peptide bond between the amino acids positioned at the mRNA on the ribosome, respectively. The tetracyclines do not exert similar effect on translation of nuclear proteins. The mitochondrial proteins encoded by the nucleus are produced at near standard rates while the few, but important, proteins encoded by the mitochondrial genome are in short supply. The skewed ratio of nuclear sourced proteins to those encoded by the mitochondrial genome creates imbalance and inefficiency in the mitochondrial matrix with a marked decrease in oxidation phosphorylation and a resulting decreased O₂ consumption.

Similar effects are observed in plants indicating that the tetracycline inhibition of mitochondrial translation is one of its basic modes of action. The mitochondria of cells in the presence of a tetracycline were smaller in size and each mitochondrion contained fewer copies of mitochondrial genome per mitochondrion. In such cells the fusion/fission balance in mitochondrial dynamics is tilted towards fission. Increased mitochondrial fission v. fusion is associated with early stages of apoptosis, a mechanism of programmed self-destruction of the cell. The fission/fusion effect was bimodal—expression of fusion genes in the cell is decreased while expression of fission genes increases. [All these are nuclear genes that encode mitochondrial proteins.]

These antibiotics are merely examples of interventions and reactions that are beneficial to cells when they occur, but that in their beneficial acts leave either transient or permanent artifacts in compromised metabolism of the affected cells, e.g., damaged genomic material, especially in mitochondrial DNA.

Many additional medications and supplements are known to impair metabolism, especially through mitochondrial activities, for example, anticonvulsants such as valproate affect the cell's well-being and metabolism by sequestering carnitine; decreasing fatty acid oxidation (β-oxidation), Krebs, ETC activity and oxidative phosphorylation and inhibiting complex IV; antidepressants, such as amitriptyline, amoxapine, fluoxetine, citalopram, etc.; antipsychotics such as chlorpromazine, fluphenazine, haloperidol, risperidone; barbiturates such as phenobarbital, Secobarbital, Butalbital, Amobarbital, Pentobarbital, etc. that increase the fission/fusion ration of mitochondria, interfere with complex I activity and inhibit mitochondrial protein synthesis; anxiety relief medications such as alprazolam, diazepam, etc.; anti-cholesterolic medications such as a statin, bile acid, ciprofibrate, etc., by reducing coenzyme Q10 availability, inhibiting the ETC, especially complex I; analgesics such as COX2 inhibitors, aspirin, ecetominophen, indomethacin, naproxen, clinoril, diclofenac, etc., by increasing oxidative stress, uncoupling oxidative phosphorylation and inhibiting the ETC; antibiotics such as quinolones, aminoglycosides, β-lactams, etc., by inhibiting β-oxidation, inhibiting mitochondrial protein synthesis; anti-arrhythmics such as amiodarone, etc., by inhibiting β-oxidation; steroids by uncoupling oxidative phosphorylation; anti-viral medications such as interferons, etc., by interfering with mitochondrial transcription; diabetic medications such as metformin, etc., by enhancing glycolysis (diversion to lactate), inhibiting oxidative phosphorylation; β-blockers by causing oxidative stress through ROS; chemotherapeutic medication such as doxorubicine, cis-platinum, tamoxifen, etc., by interfering with mitochondrial transcription; etc. As these perturbations accumulate it becomes increasing advantageous to the organism to have these accumulated “errors” expunged.

In addition to the compounds intentionally introduced into our bodies competitive biology provides additional impediments to our full functioning metabolism. Infectious bacteria attack with toxins and other defense strategies to counter our immune responses. Our microbiome is a shared relationship forcing compromises on our part. The microorganisms rapidly adapt as our diets and our metabolic responses change. One important process they use to adapt is lateral gene transfer (LGT) where fragments of genetic material are exchanged between different organisms. Bacterial resistance to antibiotics is spread from one species to another by this LGT. Bacterium-bacterium transfer is estimated to occur by the trillions in our bodies. But besides this interbacterial exchange, the prolific release from bacteria makes presence of bacterial genetic material a factor all other organisms must manage. The different organisms all share the same four nucleotide bases polymerized to form the genetic material. Each DNA molecule can be bound to another DNA molecules terminus as a simple co-polymer insertion. DNA translation and repair mechanisms can result in exposed polymer termini and if not protected allow incorporation of a foreign DNA fragment.

Such foreign DNA has been observed in genomic sequences from somatic (not ovaries or testes) human cells. Cancer cells show a higher incidence, possibly because a past insertion in a rapidly dividing cancer cell will be amplified (doubled) with each subsequent cell division and possibly because the high rate of cell divisions leaves the dividing cells especially accepting to foreign DNA fragments. All human cells (as well as cells of other organisms) have a large number of retroviral remnants in the nuclear genome, i.e., endogenous retroviruses. These are consistently present in the genomes of most individual in a species. In terminally differentiated (non-dividing) or slowly dividing cells each insertion may only appear in a single cell or a small fraction of cells. The rarity of these insertions is likely to make them appear simply as “noise” or extremely mild contamination in the sequencing data. The inserted DNA is almost certainly much more prevalent than apparent in data collections.

In fact, in a simplified experiment [D. R. Riley et al., “Bacteria-human somatic cell lateral gene transfer is enriched in cancer samples,” PLOS Computational Biology, 2013.], looking at the much smaller and therefore easier to analyze mitochondrial DNA about one third of the samples had detectable bacterial DNA inserted into the mitochondrial genome. In cancer patients, bacterial DNA was about 1000 times more likely to be found in the cancer cells as in the non-proliferating cells. However, this extreme difference in frequency may be artefactual due to the clonal nature of cancers and their rapid rates of dividing to form new cells.

The important message is that bacterial DNA gets inserted in both nuclear and mitochondrial DNA. Depending on the material inserted and location, for example (Did it interrupt an important gene or a critical gene?) the result to the cell's metabolic status may range from 1. Minimal (when a bacterial genetic fragment is small, is not expressed and if inserted is in a quiescent position in the host cell's genome); 2. Slight (bacterial fragment a little larger, slightly interferes with duplicating or reading the native genome.); 3. Moderate (bacterial gene is expressed and inhibits or competes with host metabolism; gene insertion alters transcription of a host gene (e.g., up-regulates or down-regulates transcription, results in an isoform) 4. Extensive (inserts in a position where it regulates oncogenesis or other aberrant growth). 5. Critical (bacterial insert turns on a gene or blocks a gene with results fatal to the cell).

For example in a sample of acute myeloid leukaemia cells at least a third of the inserted microbial genes were found in the mitochondrial genome. In this case the genes were predominantly from the Acinetobacter genus. In stomach cancers, Pseudomonas appeared a preferred LGT donor. In this case though nuclear DNA was a favored target with five genes especially targeted (four of these five being oncogenes!).

Another example proving the stress on the cell from LGT is seen in a 2015 report—Genome Res. 2015 Jun; 25(6): 814-824—where cancer cell sequences were found to have incorporated the host mtDNA into their nuclear DNA. A recent report from Vinodh Srinivasainagendra et al finds abnormal mtDNA incorporated into the nucleus of adenocarcinoma cells.

Viruses are better known for incorporating and expressing genomic material in host cells. Retroviruses have their RNA reversed transcribed for insertion into the nuclear DNA. Viruses, however, have multiple attack protocols on cells, often affecting mitochondria. For example many viruses including, but not limited to: hepatitis C, herpes simplex, hepatitis B, Coxackie, Epstein Barr, etc. modulate Ca⁺⁺ flux in mitochondria and often in the endoplasmic reticulum.

Some viral attacks on cell organelles are relatively specific. Hepatitis C guides delivery of the Parkin protein to the outer mitochondrial membrane leading to loss of its integrity with resultant increase in permeability and ROS generation and release. ROS attack leads to ETC reduced complex 1 activity and initiates a bulk autophagic reduction in mitochondrial numbers mass mitophagy). Removal of the damaged organelles is consistent with the survival and maintenance of HepC infected cells.

Herpes simplex 1 and 2 employ a nuclease to destroy mitochondrial DNA and the mitochondrial integrity in infected cells. Epstein Barr (EB) binds mtDNA with a single stranded binding protein inhibiting transcription and thus mitochondrial ETC activity while favoring EB replication.

To maintain genome integrity, infected cells experiencing dysfunction in their genomic expression generally begin a self-destruction process of autophagocytosis called apoptosis. A viral infection will generally trigger a ‘burglar’ alarm scenario in the cell to initiate a series of intracellular pathways that together involve a programmed cell death pathway. Many viruses have incorporated strategies to counter this defense. E.g., baculovirus includes instructions for a caspase inhibitor, p35, that is essential for its pathogenesis. Activation of cellular anti-apoptotic genes is used in some cases to convert a lytic viral infection to a persistent infection. Herpes viruses have stolen genetic material from host cells to encode a viral Bcl-2 analogue of the anti-apoptotic Bcl-2 gene present in most cells.

Post infection many viruses leave latent proteins in various cell membranes that exert lingering effects on functions including, but not limited to: transport, differentiation, kinase activation, mitochondrial fission/fusion protein balance, etc. EB takes advantage of otherwise natural antiviral and cellular defense mechanisms in a manner to promote EB survival in infected B cells. Post infection host genes including ISG20 (interferon stimulated gene), DNAJB2 (DnaJ [Hsp40] homolog and CD99), CDK8 (cyclin-dependent kinase 8), E2F2 (E2F transcription factor 2), CDK8 (cyclin-dependent kinase 8), and ACTN2 (actinin, alpha 2), exhibit lingering effects from the viral attack. Several viruses comprise coding for fragments of our native proteins. Imprecise response of our immune systems can result in autoimmune disease. Similarly a vaccine, for example from a ruptured pathogenic organism may comprise fragments similar to our native material. These may also instigate an autoimmune response. Fas is one important tunneling nanotubule associated protein whose function can prevent autoimmune responses.

These events, whether medical intervention, microbial attack, our body's natural defensive response, needed ROS production diverted to an unintended target, artifact of viral infection, etc., comprise a myriad of life events that effect chiefly mitochondria, but can have profound effects throughout the cell. Not all these errors have been characterized to allow, for example, synthesizing a specific interfering RNA to be inserted in the cell as a corrective response. In some cases, scientists have identified one or more genes, organelles or proteins whose activities compromise or are compromised. It is possible to genetically engineer responsive therapies to address the recognized dysfunction. But this strategy suffers at least two problems. First, while a primary defect may be recognized, its sequellae may not be fully appreciated and thus would be untreated. Second, the recognized defect may not be primary but a result of an upstream defect that is not as obvious, but may exert multiple downstream effects. Third, the defect may not in fact be a true defect, but in fact the optimal opportunistic response available to the cell; eliminating the defect may in fact make the situation worse. Fourth, the observed defect may only be the most obvious leaving multiple defects unaddressed.

Clearly, in the dynamism of life many things can go wrong. But our species and many others survive to this day. Our cells have adapted to these multiple stresses with responses to minimize of reverse the outside or integral assaults. This invention features methods for stimulating, redirecting or inducing the mitigation and repair tools.

Human cells have developed several robust means to maintain their survival but also to assist neighbor cells in supporting the organism's well-being. Cells assist each other and exchange information by controlling interstitial and circulatory conditions such as providing salts, nutrients, carrier proteins and cargoes, signaling switches, etc. through endocrine, circulatory, paracrine and neural, neuroendocrine, juxtracrine and lesser defined systems. Cells have also developed a mechanism though which one ell acts as a donor and provides one or more of its energy powerhouses to at least one neighbor cell with dysfunctioning mitochondria and/or other anomalies. Facilitating existing mechanisms to correct these errors, improve metabolism, including oxidative phosphorylation, β-oxidation, and other mitochondrial specialized functions, may be used to extend healthy life of organisms including human organisms.

Cell membranes are strong barriers: generally providing passage only to molecules that have, embedded in the cell membrane, a visa (a transport protein that allows a molecule to go across a membrane border) dedicated to that molecule or class of molecules. Different cells will have different visas available. So depending on cell type and the transport proteins being expressed at that instant in time, a molecule will be capable of entering some cells but not others.

Transport proteins may be quite specific in the molecules that they will recognize and carry across a membrane. Other transport proteins may be less specific, perhaps transporting molecules with an exposed amino group. And some membrane crossing can be through pores that may have size restrictions or charge restrictions, perhaps a concentrated (+) charge such as a small ion or a +2 or +3 charged molecule. Other pores may have few limitations simply providing an aqueous pore through a membrane that for the time that the pore is open allows rather free passage of any molecule that can flow through.

But there are limitations to what a pore can transport. For example, mitochondria, themselves comprise membranes with their own characteristic transport proteins. Though small in size (˜1-3 μm) in comparison to the host cell (10 s to 100 s of microns in diameter depending on type of cell and stage of development), mitochondria are magnitudes in size larger than the molecules granted passage through gated pores or by carrier proteins. Physical limitations prevent proteins (single nanometer scale in size) from forming transmembrane pores of a size and duration required to pass a substance as large as a mitochondrion. A pore of such magnitude with a diameter several magnitudes larger than the membrane thickness would theoretically cede the cell's control of ion partitioning. Mitochondria are in fact dynamic organelles present in a cell as a network of long, filamentous structures constantly moving along the intracellular highway of the cytoskeleton. Individual mitochondria may elongate or become more rodlike; they may divide in a process called fission into plural mitochondria or nearby mitochondria may fuse together to combine, share and/or exchange or replace mitochondrial components. So the cell membrane is an effective barrier to mitochondria and other similar sized organelles passing from cell to cell.

As mentioned earlier, damaged cells will undergo a process called apoptosis, a programmed orderly procedure to remove unneeded cells and rather efficiently recycle their materials. An often unappreciated but important function of mitochondria is their function in mediating intrinsic apoptosis, an energy-dependent cell death pathway that occurs in response to physiological or pathological cell stressors, such as toxins, viral infections, hypoxia, hyperthermia, free radicals, and/or DNA damage.

Apoptosis in a cell is induced by the decline in number or activity of anti-apoptotic proteins, (e.g., Bcl-2 and Bcl-XL) and/or by activation of pro-apoptotic proteins (e.g., Bax and Bak). This apoptotic pathway requires Mitochondrial Outer Membrane Permeabilization (MOMP), a critical, irreversible step that commits the cell to cytochrome c liberation and ultimately to a programmed destruction of the cell. As a direct consequence of MOMP, large amounts of cytochrome c along with other apoptogenic proteins are released from the mitochondrial inter-membrane space (space between the two mitochondrial membranes) into the cytosol, where when pro-caspace9 binds cytochrome c, caspase3 activates a cascade leading to proteolytic cleavage of intracellular proteins, DNA degradation, formation of apoptotic bodies, and other morphological changes that are considered hallmarks of apoptotic cell death. This intrinsic apoptotic pathway is essential for embryogenesis, normal growth and development, tissue remodeling, aging, wound healing, immune response, and for maintaining homeostasis in the adult human body, but when overzealous the excess cell death is a detriment to the organism's optimal functioning. The apoptotic process is a controlled process minimizing damage to surrounding cells and in fact serving as a recycling mechanism for the cell's component parts.

Every stress to a cell does not elicit an apoptotic response. Cells are tolerant of damage and have tools, either inducible or constitutive, that can ameliorate at least some of the damaging effects. Mitochondria and other organelles have several antioxidant protections, including, but not limited to: superoxide dismutase (SOD), catalase, glutathione and glutathione peroxidase, ascorbic acid, peroxiredoxins, etc. But sometimes the cell is unable on its own to restore adequate function and absent a rescue, will suffer necrosis or an apoptosis induced cell death.

Cells are capable of transference of biochemical, electrophysiological and biomolecular sub-cellular constructs and/or assemblies as carriers of material and information through continuous cytoplasmic intercourses between neighboring cells that are opportunistically provided on demand. These connections facilitate intercellular exchanges including, but not limited to: organelles, macromolecules, lipids, ions, etc. from cells able to dispose of said biomaterials, etc. to cells standing to benefit therefrom. Portions of the cells plasma membrane including receptors, pore, transport molecules, lipids, etc. Also Are transported cell to cell using TNTs. Rather than the inner tubule however, the actual tube structure that originated from plasma membrane flows from one cell to another.

As a rescue mechanism, many cells have capacity to act as first responders to construct tunneling nanotubes (TNT), a transient structure of a size compatible with in vitro organelle transfer from cell to cell as a rescue facilitator. TNTs are F-actin based long-distance cytoplasmic extensions that are several times in length and width longer that the plasma membrane thickness. Some, but not all contain microtubules. The relatively large diameter inside a TNT does not provide a strong barrier to ions or to small molecules including proteins and thus have been observed passaging a combination of water, calcium ions, major histocompatibility complex (MHC), mitochondria, the endosomal-lysosomal system, membrane segments and components, golgi complex, lysosome, ER, prions, and viral and bacterial pathogens between cells. TNTs provide continuity between the cytoplasms of non-contiguous cells allowing transfer of cytoplasmic inclusions including ions, proteins, vesicles, organelles, etc. These TNTs are labile structures that form within a few minutes for short length TNTs or up to several hours when the TNT bridges up to 5 or 6 cell diameters. TNTs may last from just a few minutes up to several hours. They may form rather transient cell-to cell connections in response to a single demand event. TNTs are also dynamic and repeatable capable of establishing a cellular and subcellular network amongst cells that may persist for days and may switch between cells to opportunistically provide the networked cells with optimal resources to meet their constantly shifting requirements.

TNTs are related to but distinct from filopodia which are exploratory, information gathering, cytoplasmic projections that are made of parallel bundles of F-actin. Filopodia formation relies on an actin nucleation complex anchored by the Rho GTPase CDC42 at the cell membrane. The end of the filopodia F-actin tip has barbed-end proteins, such as capping proteins with Ena-VASP proteins regulating actin polymerization inside the filopod. In contrast, cytonemes and TNTs are thin membrane bridges. Cytonemes are F-actin-containing cytoplasmic projections that transfer surface-associated cargoes from cell to cell. Substances bound to the outside of the membrane can proceed along the cytoneme surface to another cell. Cytoneme formation relies on specific ligand-receptor interaction between the tip of the cytoneme and the target cell. Filopodia and cytonemes are essentially solid structures, not open pipes or conduits. Because the cytoplasms are not in connection between the cells, these interconnections are non-tubular. Cytoneme outgrowth relies on a specific signal gradient triggered by the target cell. TNTs are also F-actin-based but are tubular connections with a biomembrane sealed cytoplasmic path between the separate cells. Thus cytoplasmic content: ions, proteins, vesicles, organelles and the like can pass through TNTs but not through the other structures.

In addition to ROS and immune secretions as signals that may be used individually or in combination to expedite TNTs, cardiolipin; hemeoxygenase-1; sirtuins; heat shock factors; including active fragments—including, but not limited to: HSP27, HSP40, HSP60, HSP60-HSP10, HSP70, HSP90, HSP110, etc.; heat shock factor 1, including polymers; phosphorylated eukaryotic translation initiation factor 2α; activating transcription factor 6; histone deacetylase, cytochrome c; formylated peptides; intact in interstitium, clumped, polymerized, coordinated with or bound to lipids, carbohydrates other proteins, complexes and fragments of these and similar and analogous chemical signals may also be used to expedite or excite and guide TNT initiation, growth and production. Pharmaceutical interventions are available to facilitate TNT production or to suppress TNT activities. Cannabinoids, including, but not limited to: anandamide (AEA), 2-arachidonoylglycerol (2AG), palmitoylethanolamide, noladin ether, O-arachidonoyl ethanolamine, oleoylethanolamide, plant and/or synthetic cannabinoids, including, but not limited to: cannabigerolic acid, cannabidiolic acid, Δ⁹-tetrahydrocannabinolic acid, cannabichromenenic acid, cannabigerovarinic acid, cannabichromevarinic acid, tetra-hydrocanabivarinic acid, cannabidivarinic acid, cannabigerol, Δ⁹-tetrahydrocannabinol, canna-bidivarin, cannabichromevarin, cannabichromene, cannabigerivarin, tetrahydrocannabivarin, N-isobutylamides, β-caryophyllene, pristimerin, euphol, N-acylethanolamines, Δ⁸-tetrahydro-cannabinol, guineensine, capsaicin, resiniferatoxin, HU-210, HU-331, JWH015, SATIVEX™ or its generic, dronabinol, nabilone, ajulemic acid, CP 55 940, CANNABINOR™ or its generic, methan-andamide, THC-11-oic acid, TARANABANT™ or its generic, etc., may act in conjunction with sirtuins; HSPs, e.g., HSP90 (a CB₂ chaperone), or through TRPV1 to upregulate HSP27, HSP70, HSP90, etc. Any of these compounds or fragments may be provided intact, as a compound cleavable from a carrier portion, or attached to a carrier portion. One or more methylene groups —CH₂— may be incorporated internally within the carbon chains to alter effects including, but not limited to: selectivity ratio for alternate receptors, half-life of activity, rate of delivery, level of activity, toxicity, cost of production, etc.

In several instances molecular interactions of cannabinoid compounds with effector pathways for potentiating TNT formation are established. In many cases however, the precise pathway remains to be characterized. The pathways and interactions suggested herein relate to current understanding whose knowledge is not essential for practicing the invention. The suggested interactions are incorporated herein for guided understanding and should not be understood as dictum or as stages required for practicing the invention. The skilled artisan will also understand that these interventions are dose dependent. At therapeutic dosages affecting compromised cells with a reduced threshold for eliciting a distress call, less compromised cells will not see their thresholds breached; the needy cells will entreat one or more enabled neighbor cells for rescue through TNTs and/or other means. However, at higher dosages, the intervention(s) prompting the distress signal(s) would be expected to goad comprised cells to undergo actions associated with a poorer outcome, for example signaling their initiation of apoptosis. Signals resulting from these excessive dosages would have no reason to cause a TNT response.

Ascorbic acid (vitamin C) can be used in the presence of activating cannabinoids to potentiate activity of CB₁·H⁺ and high glucose increase TNTs. Growth factors including, but not limited to: EGF, FGF, TGFB-3, etc. and their analogues or active fragments thereof are additional tools that may be delivered to interstitial areas or neighboring cells to accentuate TNT formation. The antibiotic, zeocin is also usable as a TNT stimulator. Delivery of these activators includes delivering as a portion of a molecule, whose delivery portion may remain attached or be partially or entirely cleaved to present the activity. These activators may be used individually or in combination with other active or carrier substance(s).

Passing mitochondria from one cell to another cell may have several effects. The passaged mitochondria may replace non-functioning mitochondria of the recipient cell. But even mitochondria that appear to function in energy production and other mitochondrial metabolic tasks my threaten cell survival. Even if the mitochondria of a cell are synthesizing ATP, survival of the cell may be incompatible with its mitochondria. For example, mitochondria are notorious for their epigenetic influence on the host cell's genome, not just for transcription and translation of mitochondrial proteins, but of many non-mitochondrial attached genes that are involved in metabolism. When epigenetic influences are transient or reversible they may be corrected using mitochondrial re-engineering when achieved molecularly by the cell or by mitochondrial transfer to optimize epigenetic status.

To establish a continuous open ended TNT connection, membrane fusion needs to occur between the nascent TNT tip and the targeted cell. Since the lipid bilayers have charged hydrophilic/lipophobic surfaces, merging of two lipid bilayers requires disruptive energy to allow continuity of the membranes. This energy can be provided by fusion molecules, such as SNARE proteins or in some pathologic cases, viral fusion proteins. These or alternative membrane-fusion molecules located at the tip of the nascent TNT can be used for breaching the targeted cell's membrane barrier. In some cases, the lipid bilayer composition will favor membrane fusion because, depending on its molecular structure, some lipids adopt a specific curvature that in the right environment, e.g., ionic strength, nearby lipid influences, results in spontaneous membrane fusion. The lipid composition at the end of the TNT when it provides a high degree of curvature will provoke spontaneous membrane fusion between the TNT tip and the targeted cell.

TNTs can also arise by a process analogous to that of filopodia, that is, with actin involvement in guiding the tubular formation. Actin when attached to a membrane generally binds through the C-terminus of ezrin, a membrane associated protein, either in a cytoskeletal context or when involved in a protrusion.

TNT initiation and growth can be induced using, for example intact or active fragments of, immune system cytokines and/or mimetics or biosimilars and/or one or more cell distress signal, either induced for endogenous production at the site or added from an extraneous source individually or as part of a combination cocktail. For example, tumor necrosis factor, as an example of one distress signal protein, can be used to initiate outgrowth towards a target cell. Macrophage M-sec protein can also be employed in the initiation and growing process when it interfaces with Ral-A, a filamin binding protein that cross-links actin strands. TNT length is controllable using CD42 which when bound to GDP limits TNT length. In the presence of proper nutrition and lipid supply M-Sec promotes membrane formation thereby providing raw structural material for the TNT tube. In at least one cell class, such as T cells, stimulation of Fas though a Rho GTPase induces TNT formation and elongation. Histone deacetylase inhibitors promote TNT rescue especially promoting TNTs whose intended cargo is mitochondria. Analogous proteins or biosimilars may be used in place of any of these native proteins for the same effect.

TNTs may also appear in proximity to filopodia. The filopodic connection establishes a linkage holding the cells in proximity to one another which, because of the reduced distance, results in a lowered initiation threshold necessary to carry out the growth and maintenance of the TNT. TNTs may result after filopodia form and then retract leaving the TNT connection between the cells.

Hydrogen peroxide (and possibly other oxidative stressors) will act through p53 initiated pathways cascading to form TNTs. This mechanism may on rare occasions allow emptying of severely stressed cells, but generally is a process whereby recoverable cells reach out to neighbors to obtain undamaged repair components. The stressed cell reaches out to a neighbor cell by producing a gradient, e.g., through release of H⁺, H₂O₂ or another cytoactivator. The responding neighbor donor cell then initiates TNT growth which continues along the signal gradient to the stressed recipient cell. The TNT apex then merges with the stressed cell's plasma membrane to form a continuous passage through which ions, biomolecules and/or organelles can enter the recipient cell to effect its recovery. One or more gradients including, but not limited to: a temperature gradient, an electrochemical gradient, an osmotic gradient, an ionic strength gradient, a pressure gradient, a magnetic gradient and/or an electric field gradient can be exogenously initiated or applied to drive TNT formation.

NK cells and in some cases macrophages build a special form of TNT, one with microtubules. These are similar in polymerization effect and in some manners are reminiscent of intracellular protein bridges. In both microtubule based and F-actin based structures, Ca⁺⁺ binding is involved in the actomyosin within the TNT actively driving or transported cargo step by step, as the Ca^{++ in} binds and unbinds the actin, through the TNT from one cell to the other. The process is directed and rapid i.e., significantly more rapid than diffusion. The requirement for Ca^{++ in} transport results in Ca^{++ in} flux generally along its concentration gradient which can be controlled by IP3 release of Ca^{++ in} from the ER or by alternative means of increasing Ca⁺⁺ concentration. One function of TNTs is to provide connections forming a network of multiple cells such that when one cell is stimulated and its cytoplasmic Ca^{++ in} increases, this Ca^{++ in} activation is rapidly spread throughout the network in a process somewhat akin to a neural network but without neurotransmitter involved for the cell to cell activation. Heat, pH, hypoxia, and/or chemical and/or biochemical signaling agents may be advantageously applied in isolation or combination to expedite exchange between cells. Intercellular feedback may cause TNT switching events follow a harmonic cycle.

The direct connection that TNTs provide between cells allows electrical propagation directly from one cell to another absent a synapse as used for cell-to-cell information transfer in the neural system. This direct electrical connection aids in connecting cells at the leading edge of a healing wound; and also can be used to repair metabolically compromised cells surrounding or surrounded by healthy cells. Healthy cells may be cells native to the organism and originally at that location. They may be cells native to the organism but driven using one or more chemotactic factor to the region to be healed. They may be cells native to the organism, but removed and cultured in growth or restorative media before return to the organism to aid healing. Or the cells may be immunologically compatible cells cultured from another source and provided as an aid to healing. The healing cells with their direct electrical connections may exert their effects by activating enzymes, such as voltage-sensitive phosphatase, P₁₃K and protein kinase A.

The direct electrical connection through TNTs allows a population of cells to act as a biological swarm computing system with gating strength determined by cell location, number of connections to a neighbor cell, number of cells connected to, spatial position (determining timing and additive effects), size and content of TNTs, etc. Although less rapid material flux between these connected cells is available to act as a reprogramming mechanism, for example, providing organelles, enzymes, substrate, transcription factors, epigenetic modifiers etc. Even in cases when a transcription factor may have been epigenetically modified in one cell, downstream effect is restorable through these TNTs.

In circumstance where a cell is comparatively worse off and a candidate for autophagocytosis or better positioned than neighbor cells for disposing wastes, the TNTs can be used for discarding non-functioning or otherwise disposable biomaterials. This is the opposite of the restorative effect with first responder helper cells that deliver prime pieces to the needy cells. In this arrangement a cell that may have signaled initiation of apoptosis allows neighboring cells to advantageously “dump” excess, outdated, or waste materials to the cell about to undergo apoptosis. This allows for efficient disposal of wastes and improved functioning of many cells with only the death of the one cell. A variation of this action, such as for intestinal cells when cells at or near the lumen are better positioned to dispose of wastes, involves at least two cells but preferably a network of cells that periodically form TNT connections to deliver their discards to the cell best positioned to eliminate them. The TNTs may form under diurnal control and be inducible by controlling adenosine or other fluctuating metabolites or hormones. Fluctuations, including fluctuations in number, size, flow direction, materials exchanged, etc. may be under a cyclic, (e.g., meal based, diurnal, lunar, weekly) control, may be harmonic relying on a cycling stimulus or simple or multi-stage feedback loop arrangement.

The TNT mechanism can be used to deliver specially selected or engineered mitochondria or mitochondrial like vesicular components to aged, diseased or otherwise compromised cells. Donor cells maybe cultured for robust health including a supply of optimally functioning mitochondria or may be engineered to comprise mitochondria or mitochondrial-like vesicles. Mesenchymal or other cells can be modified to comprise such engineered mitochondria or vesicles. The engineering may involve nuclear engineering to provide engineered mitochondrial proteins, may involve mitochondrial or vesicular insertion and/or may involve engineered mitochondrial genome. Even when these engineered donations are transitory, the healing effect may produce durable desired results. The donor cell and mitochondrial material can be specially chosen for a transitory survival or toxic switch characteristic to reduce the risk of or to have ability to switch off future long-term actions.

Cells in a compromised metabolic status with increased lactate release, lead to an environment where interstitial pH is reduced. The increased H⁺ ion concentration is one available signal for inducing TNT formation. An acidic environment thus may be used to augment wound healing and/or to increase intercellular repair (and/or replacement) processes.

Although TNTs are available for their healing qualities, the TNT mechanism might also be employed to the organism's detriment. For example, if TNTs are induced during a cancer therapy, it is possible that therapeutic effectiveness could be reduced since increased TNT activity might aid healing and maintenance of the cancer cells under chemo-attack. In such instances, promotion and inhibition of TNTs are preferably coordinated with other therapeutic interventions, including inhibited TNT activity during chemotherapeutic, radiation or other cancer cell damaging events.

Alternatively, the request by the tumor cells under chemo or radiation attack to harness TNTs for restoration and healing from the therapy can be turned on the requesting cells. Cells that are cultured for responding to the tumor cell's requests for TNT assistance can be delivered to the tumor by methods known in the art, for example, targeted injection using imaging (live or historical), tactile targeting when the tumor is palpable, injection into blood vessel supplying the tumor, use of an injection gun, incorporating a receptor or receptor ligand into the cultured cell that is specific to or preferentially recognized by the tumor plasma membrane or attracted to a secretion from the tumor, injecting a chemoattractant into the tumor that harvests the cultured cells from more general circulation, multiple injections into the tumor mass, implantation of a capsule in the tumor or vicinity, etc. The cells may be engineered cells with defective organelles, e.g., mitochondria. The defect is overcome by supplementing the culture medium allowing the cells to reproduce. However, when deprived of the supplement the mitochondria lack essential components and therefore are unable to rescue the cells being treated. Multiple alternative approaches are available. Engineered cells may comprise a slow release or enzyme active toxin. Especially preferred are toxins preferentially activated by the tumor. The “rescue” cells may be infected with injected or otherwise supplemented with a virus effective against the tumor cell. Components that will further sensitize the tumor cells to chemo or radiative attack provide additional alternatives. Varied options are available that may be used individually, sequentially or in a parallel. In sequential strategies, multiple tools for attacking or weakening the tumor cells may be used at any or all planned stages. The rescue need not be intracellular. Since membrane materials of the TNT membrane are available for sharing, a substance, e.g., a lipid substance, a membrane protein, a membrane lipoprotein, a lipid dye or other marker, etc., may be incorporated into the “rescue” membrane. The incorporated material may be a material that on its own, e.g., upon exposure to a targeting chemical, ultrasound frequency, targeting macromolecule, may have detrimental effect on the cell. The incorporated material may, for example, act as a conduit or facilitator for a toxin. For example, the incorporated material may be a membrane receptor, a pore, a transport protein, etc., that is a form of toxin facilitator substance which allows a detrimental substance to attach to or enter into the tumor cell. Rather than acting as a savior to the cell that sent one or more distress signals requesting aid, the engineered rescue cells continue the cytotoxic process helping to eliminate or shrink the tumor.

Throughout this application biologic materials may be identified by protein name or by its gene. Genes encoding the same or analogous protein or biosimilar are considered encompassed in the definition of the named gene. Likewise protein mimetics, active fragments, proteins encoded by analogous genes, analogous proteins, protein mimetics and the like are encompassed in the definition of the named protein.

1. A method to improve health of an individual, said method comprising: providing to an individual whose metabolism in at least one cell is stressed or compromised, at least one chemical component capable of stimulating the at least one stressed or compromised cell to signal at least one neighbor cell to initiate a TNT bridging process for delivery of stress mitigating or compromise curing material to said stressed or compromised cell.

2. The method of claim 1 wherein said at least one chemical component is selected from the group consisting of: H⁺, H₂O₂, zeocin, TNT inducing growth factor, histone deacetylase inhibitor, a compound that in the presence of said at least one stressed or compromised cell increases H⁺ concentration, and a compound that in the presence of said at least one stressed or compromised cell increases H₂O₂ concentration.

3. The method of claim 2 wherein said at least one chemical component is selected from the group consisting of: EGF, FGF and TGFB-3.

4. The method of claim 2 wherein said at least one chemical component is selected from the group consisting of: cardiolipin; hemeoxygenase-1; sirtuins, heat shock factor, phosphorylated eukaryotic translation initiation factor 2α, activating transcription factor 6, cytochrome c, and formylated peptides.

5. The method of claim 4 wherein said at least one chemical component is selected from the group consisting of: HSP27, HSP40, HSP60, HSP60-HSP10, HSP70, HSP90, HSP110, and heat shock factor 1.

6. The method of claim 4 wherein said at least one chemical component in a format selected from the group consisting of: intact in interstitium, clumped, polymerized, coordinated with or bound to lipids, carbohydrates other proteins, complexes and fragments of these and similar and analogous chemical signals.

7. The method of claim 4 wherein said at least one chemical component is selected from the group consisting of: anandamide (AEA), 2-arachidonoylglycerol (2AG), palmitoylethanolamide, noladin ether, O-arachidonoyl ethanolamine, oleoylethanolamide, plant cannabinoid, and synthetic cannabinoid.

8. The method of claim 7 wherein said at least one chemical component is selected from the group consisting of: cannabigerolic acid, cannabidiolic acid, Δ⁹-tetrahydrocannabinolic acid, cannabichromenenic acid, cannabigerovarinic acid, cannabichromevarinic acid, tetrahydrocanabivarinic acid, cannabidivarinic acid, cannabigerol, Δ⁹-tetrahydrocannabinol, cannabidivarin, cannabichromevarin, cannabichromene, cannabigerivarin, tetrahydrocannabivarin, N-isobutylamides, β-caryophyllene, pristimerin, euphol, N-acylethanolamines, Δ⁸-tetrahydrocannabinol, guineensine, piperoleine B, piperdardine, capsaicin, resiniferatoxin, HU-210, HU-331, JWH015, SATIVEX™ or its generic, dronabinol, nabilone, ajulemic acid, CP 55940, CANNABINOR™ or its generic, THC-11-oic acid, TARANABANT™ and methanandamide.

9. The method of claim 1 further comprising providing to said individual, at least one nutrition element targeted for improving at least one stressed or compromised cell's mitochondria's current metabolic status.

10. The method of claim 9 wherein said at least one nutrition element is selected from the group consisting of: a substrate, cofactor, or enzymatic activity modulator.

11. The method of claim 10 wherein said at least one nutrition element is selected from the group consisting of: Riboflavin (B₂), L-Creatine, CoQ₁₀, L-arginine , L-carnitine, vitamin C, cyclosporin A, manganese, magnesium, carnosine, vitamin E, resveratrol, α-lipoic acid, folinic acid, fulvic acid, dichloroacetate, succinate, a prostaglandin (PG), a prostacyclin, a thromboxane, prostanoic acid, 2-arachidonoylglycerol, an NSAID, melatonin, cocaine, amphetamine, AZT, a mitophagic controlling compound, glutathione, β-carotene and a different carotenoid.

12. A method for increasing apoptosis following cellular stress, said method comprising delivering to a vicinity of a stressed cell at least one chemical component capable of stimulating formation of tunneling nanotubules that are capable of interconnecting at least two cells and allowing passage of intracellular materials between the cells, said intracellular material comprising at least one component selected from the group consisting of: a cytotoxin, a proapoptotic component, and a toxic effect facilitator.

13. A method of delivering specially selected or engineered mitochondria or mitochondrial-like vesicular components to aged, diseased or otherwise compromised cells comprising: making use of at least one tunneling nanotubule (TNT) interconnecting at least two cells to deliver a mitochondrion or a mitochondrial-like vesicular component from a donor cell to a recipient cell that would benefit therefrom.

14. The method of claim 10 wherein said donor cell is a cell cultured to comprise optimally healthy mitochondria or a cell modified to comprise an engineered mitochondrion or a mitochondrial-like vesicle.

15. The method of claim 14 wherein at least one said donor cell is a mesenchymal cell.

16. The method of claim 3 wherein at least one said donor cell comprises results of nuclear engineering evident in mitochondrial structure.

17. The method of claim 14 wherein at least one said donor cell comprises results of mitochondrial genome engineering evident in a mitochondrial protein or expression of at least one protein extraneous to the mitochondrion.

18. The method of claim 14 wherein the donor cell comprises a transitory donated packet.

19. The method of claim 18 wherein the transitory donated packet comprises a toxicity switch having a characteristic permitting its activation to destroy at least some effect of the donation.

20. The method of claim 1 wherein said at least one neighbor cell comprises a cell attracted to a proximity that surrounds said at least one stressed or compromised cell by use of at least one gradient.

21. The method of claim 20 wherein at least one gradient is selected from the group consisting of: a chemical gradient, temperature gradient, an electrochemical gradient, an osmotic gradient, an ionic strength gradient, a pressure gradient, a magnetic gradient and an electric field gradient.

22. The method of claim 1 wherein said at least one neighbor cell comprises a cell native to the organism, but removed and cultured in growth or restorative media before return to the organism.

23. The method of claim 1 wherein said at least one neighbor cell comprises an immunologically compatible cell cultured from another source prior to becoming said neighbor cell.

24. The method of claim 1 wherein at least one exogenous chemical compound is associated with the stress or compromise affecting the at least one cell.

25. The method of claim 24 wherein said at least one exogenous chemical comprises a compound selected from the group consisting of: an anticonvulsant, an antidepressant, an antipsychotic, a barbiturate, an anxiety relief medication, an anti-cholesterolic medication, an analgesic, an antibiotic, an anti-arrhythmic, a steroid, an anti-viral medication, a vaccine, a diabetic medication, a beta-blocker and a chemotherapeutic medication.

26. The method of claim 1, wherein at least one infectious particle is associated with the stress or compromise affecting the at least one cell.

27. The method of claim 26 wherein said at least one infectious particle is selected from the group consisting of a prion, a virus and a bacterium.

28. The method of claim 1 wherein said at least one chemical component comprises a biochemical.
